(19) United States
(12) Patent Application Publication
(10) Pub. No.: US 2015/0208924 A1
(43) Pub. Date: Jul. 30, 2015

(54) PHOTOACOUSTIC SENSORS WITH DIFFUSING ELEMENTS FOR PATIENT MONITORING

(71) Applicant: COVIDIEN LP, MANSFIELD, MA (US)

(72) Inventors: Youzhi Li, Longmont, CO (US); Qiaojian Huang, Broomfield, CO (US); Sarah L. Hayman, Boulder, CO (US); Charles Keith Haisley, Boulder, CO (US)

(73) Assignee: COVIDIEN LP, MANSFIELD, MA (US)

(21) Appl. No.: 14/165,224

(22) Filed: Jan. 27, 2014

(51) Int. Cl. A61B 5/00

(52) U.S. Cl. CPC A61B 5/0095

(57) ABSTRACT

Various methods and systems for photoacoustic patient monitoring are provided. A photoacoustic system includes a sensor having a body and one or more light emitting components disposed in the body that emit one or more wavelengths of light. A light guide receives the one or more wavelengths of light and guides the one or more wavelengths of light into an interrogation region of a patient. A wide angle diffusing element is disposed on a tip of the light guide and has a diffusing angle of greater than or equal to 80 degrees. The wide angle diffusing element diffuses the one or more wavelengths of light when the sensor is opposed by air. One or more acoustic detectors are disposed in the body and detect acoustic energy generated by the interrogation region of the patient in response to the emitted light when the sensor is opposed by the patient.

### BACKGROUND

The present disclosure relates generally to medical devices and, more particularly, to the use of photoacoustic sensors in patient monitoring.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological characteristics of their patients. Accordingly, a wide variety of devices have been developed for monitoring patient characteristics. Such devices provide doctors and other healthcare personnel with the information they need to provide healthcare for their patients. As a result, such monitoring devices have become an indispensable part of modern medicine. Further, in certain medical contexts, it may be desirable to ascertain various localized physiological parameters, such as parameters related to individual blood vessels or other discrete components of the vascular system. Examples of such parameters may include oxygen saturation, hemoglobin concentration, perfusion, and so forth, for an individual blood vessel.

In one approach, measurement of such localized parameters is achieved via photoacoustic (PA) spectroscopy. PA spectroscopy utilizes light directed into a patient's tissue to generate acoustic waves that may be detected and resolved to determine localized physiological information of interest. In particular, the light energy directed into the tissue may be provided at particular wavelengths that correspond to the absorption profile of one or more blood or tissue constituents of interest. In some systems, the light is emitted as pulses (i.e., pulsed PA spectroscopy), though in other systems the light may be emitted in a continuous manner (i.e., continuous PA spectroscopy). The light absorbed by the constituent of interest results in a proportionate increase in the kinetic energy of the constituent (i.e., the constituent is heated), which results in the generation of acoustic waves. The acoustic waves may be detected and used to determine the amount of light absorption, and thus the quantity of the constituent of interest, in the illuminated region. For example, the detected ultrasound energy may be proportional to the optical absorption coefficient of the blood or tissue constituent and the fluence of light at the wavelength of interest at the localized region being interrogated (e.g., a specific blood vessel).

In many PA systems, a high intensity light emitter, such as a laser, is chosen to provide enough optical power and power density to excite the patient's tissue, such as the blood present in the patient's blood vessel, and to generate a large enough acoustic signal that can be detected with a desired signal-to-noise ratio. Unfortunately, while higher intensity light emitters may give rise to improved signal-to-noise ratios, industry standards may involve additional equipment with such light sources, such as operator use of light-shielding goggles, when using these emitters in a clinical setting. Accordingly, there exists a need for PA systems and methods that enable a high signal-to-noise ratio PA signal to be obtained without the drawbacks typically associated with the use of high intensity light emitters.

### BRIEF DESCRIPTION OF THE DRAWINGS

Advantages of the disclosed techniques may become apparent upon reading the following detailed description and upon reference to the drawings in which:

FIG. 1 is a block diagram of a patient monitor and photoacoustic sensor in accordance with an embodiment;

FIG. 2 illustrates a cross sectional view of a photoacoustic sensor assembly having a diffusing element in accordance with an embodiment;

FIG. 3 illustrates an exploded view of the photoacoustic sensor assembly of FIG. 2;

FIG. 4 is a chart illustrating conditions for measuring and determining classification of a laser according to the IEC 60825-1 standard;

FIG. 5 is a diagrammatical representation of a setup used to test the photoacoustic sensor assembly of FIGS. 2 and 3 in accordance with an embodiment;

FIG. 6 is a chart illustrating experimental results obtained for an embodiment of the photoacoustic sensor assembly of FIGS. 2 and 3 when tested in accordance with the chart of FIG. 4; and

FIG. 7 is a plot of an example photoacoustic signal generated with a photoacoustic sensor having a diffusing element and an example photoacoustic signal generated with a photoacoustic sensor not having a diffusing element in accordance with an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, presently disclosed embodiments of PA sensors, systems, and methods are provided for the measurement of various localized physiological parameters, such as parameters related to individual blood vessels or other discrete components of the vascular system. Examples of such parameters may include but are not limited to oxygen saturation, regional saturation, hemoglobin concentration, perfusion, cardiac output, and so forth, for an individual blood vessel. Certain features of the disclosed embodiments may reduce the laser safety class to which the PA sensor is assigned, thus reducing the mandated safety measures that must be taken in a clinical environment while maintaining the optical power and power density desired to improve the likelihood that a blood PA signal in a patient's blood vessel will be distinguishable in the acquired measurement.

In certain embodiments, the disclosed PA sensors may be utilized as part of a PA spectroscopy system in which light is directed into a patient's tissue to generate acoustic waves that may be detected and resolved to determine the localized physiological information of interest. In these embodiments, the light energy directed into the tissue is provided at particular wavelengths that correspond to the absorption profile of one or more blood or tissue constituents of interest. Disclosed embodiments may be utilized in PA spectroscopy systems in which the light is emitted as pulses (i.e., pulsed photoacoustic spectroscopy), as well as in systems in which the light is emitted in a continuous manner (i.e., continuous photoacoustic spectroscopy). In disclosed embodiments, once the light is emitted into the patient, the acoustic waves may be detected with an ultrasound transducer or transducer array, which may be made, for example, of piezoelectric materials such as lead zirconate titanate (PZT), polyvinylidene fluoride (PVDF), and so forth.

In one disclosed embodiment, the PA sensor uses a pulsed Class 3B laser (as defined by the IEC 60825-1 standard) that provides enough optical power to excite the blood present in the patient's blood vessel at a level sufficient to generate an acoustic signal with a desired signal-to-noise ratio. However, the PA sensor assembly is reduced to a Class 1 laser by placing a wide angle diffuser on the tip of the optical fiber light delivery system such that the light emitted by the light source is diffused when the PA sensor is opposed by air. The foregoing feature of this embodiment may reduce the use of associated equipment (e.g., light-shielding goggles) when the PA sensor is utilized in a clinical setting while maintaining the desired signal-to-noise ratio. That is, as described in more detail below, embodiments disclosed herein may enable PA sensor assemblies that reduce the laser safety classification of the total sensor assembly as compared to the classification of only the light emitting element used therein while maintaining the ability to acquire a clinically desirable signal.

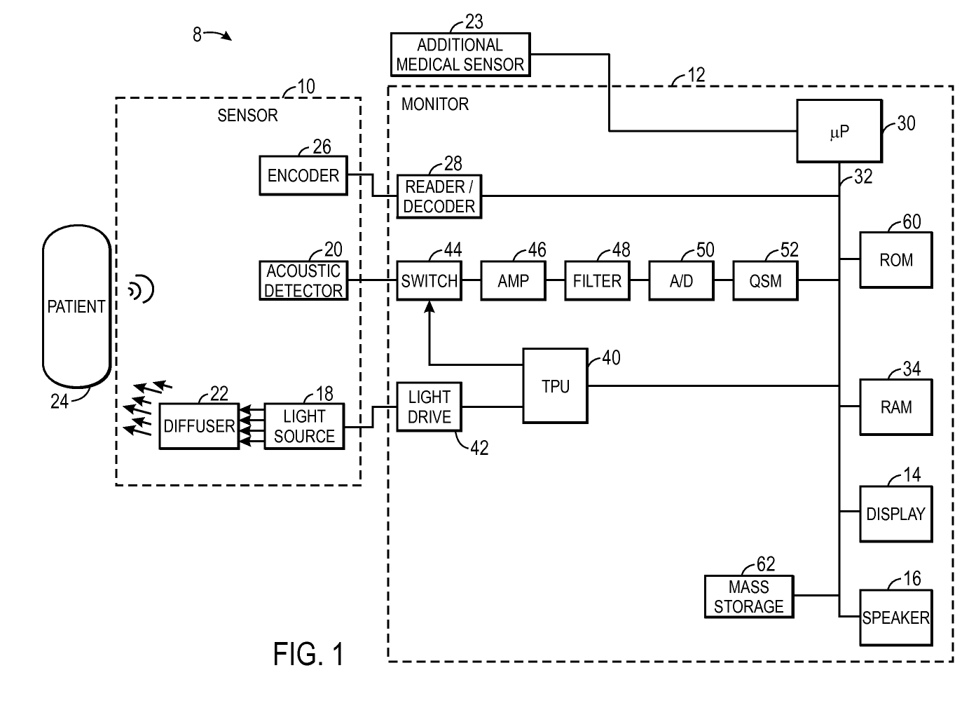

With this understanding, FIG. 1 depicts a block diagram of a photoacoustic spectroscopy system 8 in accordance with embodiments of the present disclosure. The system 8 includes a photoacoustic spectroscopy sensor 10 and a monitor 12. During operation, the sensor 10 emits spatially modulated light at certain wavelengths into a patient's tissue and detects acoustic shock waves generated in response to the emitted light. The monitor 12 is capable of calculating physiological characteristics based on signals received from the sensor 10 that correspond to the detected acoustic shock waves. The monitor 12 may include a display 14 and/or a speaker 16, which may be used to convey information about the calculated physiological characteristics to a user. The sensor 10 may be communicatively coupled to the monitor 12 via a cable or, in some embodiments, via a wireless communication link.

In one embodiment, the sensor 10 may include a light source 18 and an acoustic detector 20, such as an ultrasound transducer. The present discussion generally describes the use of laser light sources, such as pulsed light sources or continuous wave light sources in other embodiments. However, it should be understood that other high intensity light sources may also be used in conjunction with the sensor 10. Further, in certain embodiments, the light source 18 may be associated with one or more optical fibers for conveying light from one or more light generating components to the tissue site.

In the illustrated embodiment, the light source 18 is associated with a diffusing element 22 that diffuses the light from the light source 18 when the sensor 10 is opposed by air. The diffusing element 22 may be provided in a variety of forms and locations with respect to the components of the sensor 10, depending on implementation-specific considerations. For example, in certain embodiments, the light source 18 may be an optical fiber, and the diffusing element 22 may be integrated with the tip of the optical fiber, deposited onto the tip of the optical fiber, adhered to the tip of the optical fiber, or incorporated with the optical fiber in any other suitable manner. In other embodiments, the exit surface of the optical fiber may include micro-scale or nano-scale roughness sufficient to diffuse the light from the light source 18. Further, in some embodiments, a protective layer or coating may be placed over the diffusing element 22 to reduce or prevent the likelihood that the diffusing element 22 will wear off (or reduce the rate at which the diffusing element 22 does so) or become unattached from the optical fiber during use.

Still further, in some embodiments, the diffusing element 22 may be a wide angle diffuser that diffuses the light from the light source 18 at a predetermined wide angle. For example, in one embodiment, the wide angle diffuser may diffuse light at an angle greater than or equal to approximately 80 degrees. The foregoing feature may be advantageous in clinical implementations in which the sensor 10 is utilized in combination with a conductive ultrasonic gel, which is employed for the purpose of coupling an ultrasonic transducer to a patient's tissue. For example, use of a wide angle diffuser having a diffusing angle of at least 80 degrees enables the aforementioned benefits of utilizing the diffusing element 22 to be realized even though the diffusing element 22 causes the generated beam spot to have increased uniformity, and the ultrasonic gel causes the emitted light to have an increased intensity. That is, presently disclosed systems and methods enable use of a diffusing element 22 to reduce the classification of the light source 18 while maintaining compatibility with ultrasonic gels.

Accordingly, in some embodiments, one or more features of the diffusing element 22 may be selected that enable the diffusing element 22 to diffuse the light emitted by the light source 18 to be classified in Class 1 according to the International Electrotechnical Commission (IEC) 60825-1 standard when the interface of the sensor 10 that opposes the patient 24 is opposed by air, and to be classified as a Class 1 according to the IEC 60825-1 standard when the interface is coated with a conductive ultrasonic gel and the gel is opposed by air. For example, the diffusing angle of the diffusing element 22 may be selected to be greater than or equal to 80 degrees.

Further, the light source 18 and the acoustic detector 20 that may be of any other type suitable for a desired application. For example, in one embodiment, the light source 18 may include one, two, or more light emitting components (such as light emitting diodes) adapted to transmit light at one or more specified wavelengths before the light is diffused by the diffusing element 22. In certain embodiments, the light source 18 may include a laser diode or a vertical cavity surface emitting laser (VCSEL). The laser diode may be a tunable laser, such that a single diode may be tuned to various wavelengths corresponding to a number of different absorbers of interest in the tissue and blood. That is, the light may be any suitable wavelength or wavelengths (such as a wavelength between about 500 nm to about 1000 nm or between about 600 nm to about 900 nm) that are absorbed by a constituent of interest in the blood or tissue. For example, wavelengths between about 500 nm to about 600 nm, corresponding with green and yellow visible light, may be absorbed by deoxyhemoglobin and oxyhemoglobin. In other embodiments, red wavelengths (e.g., about 600 nm to about 700 nm) and infrared or near infrared wavelengths (e.g., about 800 nm to about 1000 nm) may be used. In one embodiment, the selected wavelengths of light may penetrate into the tissue of the patient 24 up to approximately 1 cm to approximately 2 cm.

The emitted light may be intensity modulated at any suitable frequency, such as from 0.1 MHz to 10 MHz or more (e.g., at 0.5 MHz). In one embodiment, the light source 18 may emit pulses of light at a suitable frequency where each pulse lasts 10 nanoseconds or less and has an associated energy of a 1 mJ or less, such as between 1 μJ to 1 mJ. In such an embodiment, the limited duration of the light pulses may prevent heating of the tissue while still emitting light of sufficient energy into the region of interest to generate the desired acoustic waves when absorbed by the constituent of interest.

In one embodiment, the acoustic detector 20 may be one or more ultrasound transducers suitable for detecting ultrasound waves emanating from the tissue in response to the emitted light and for generating a respective electrical signal in response to the ultrasound waves. For example, the acoustic detector 20 may be suitable for measuring the frequency and/or amplitude of the acoustic waves, the shape of the acoustic waves, and/or the time delay associated with the acoustic waves with respect to the light emission that generated the respective ultrasound waves. In one embodiment an acoustic detector 20 may be an ultrasound transducer employing piezoelectric or capacitive elements to generate an electrical signal in response to acoustic energy emanating from the tissue of the patient 24, i.e., the transducer converts the acoustic energy into an electrical signal.

In some embodiments, the system 10 may also include any number or combination of additional medical sensors 23 or sensing components for providing information related to patient parameters that may be used in conjunction with the PA spectroscopy sensor 10. For example, suitable sensors may include sensors for determining blood pressure, blood constituents, respiration rate, respiration effort, heart rate, patient temperature, cardiac output, and so forth.

In one embodiment, the photoacoustic sensor 10 may include a memory or other data encoding component, depicted in FIG. 1 as an encoder 26. For example, the encoder 26 may be a solid state memory, a resistor, or combination of resistors and/or memory components that may be read or decoded by the monitor 12, such as via reader/decoder 28, to provide the monitor 12 with information about the attached sensor 10. For example, the encoder 26 may encode information about the sensor 10 or its components (such as information about the light source 18 and/or the acoustic detector 20). Such encoded information may include information about the configuration or location of photoacoustic sensor 10, information about the type of lights source(s) 18 present on the sensor 10, information about the wavelengths, pulse frequencies, pulse durations, or pulse energies which the light source(s) 18 are capable of emitting, information about the nature of the acoustic detector 20, and so forth. This information may allow the monitor 12 to select appropriate algorithms and/or calibration coefficients for calculating the patient's physiological characteristics, such as the amount or concentration of a constituent of interest in a localized region, such as a blood vessel.

In one embodiment, signals from the acoustic detector 20 (and decoded data from the encoder 26, if present) may be transmitted to the monitor 12. The monitor 12 may include data processing circuitry (such as one or more processors 30, application specific integrated circuits (ASICS), or so forth) coupled to an internal bus 32. Also connected to the bus 32 may be a RAM memory 34, a speaker 16 and/or a display 14. In one embodiment, a time processing unit (TPU) 40 may provide timing control signals to light drive circuitry 42, which controls operation of the light source 18, such as to control when, for how long, and/or how frequently the light source 18 is activated, and if multiple light sources are used, the multiplexed timing for the different light sources.

TPU 40 may also control or contribute to operation of the acoustic detector 20 such that timing information for data acquired using the acoustic detector 20 may be obtained. Such timing information may be used in interpreting the shock wave data and/or in generating physiological information of interest from such acoustic data. For example, the timing of the acoustic data acquired using the acoustic detector 20 may be associated with the light emission profile of the light source 18 during data acquisition. Likewise, in one embodiment, data acquisition by the acoustic detector 20 may be gated, such as via a switching circuit 44, to account for differing aspects of light emission. For example, operation of the switching circuit 44 may allow for separate or discrete acquisition of data that corresponds to different respective wavelengths of light emitted at different times.

In one embodiment, the received signal from the acoustic detector 20 may be amplified (such as via amplifier 46), may be filtered (such as via filter 48), and/or may be digitized if initially analog (such as via an analog-to-digital converter 50). The digital data may be provided directly to the processor 30, may be stored in the RAM 34, and/or may be stored in a queued serial module (QSM) 52 prior to being downloaded to RAM 34 as QSM 52 fills up. In one embodiment, there may be separate, parallel paths for separate amplifiers, filters, and/or A/D converters provided for different respective light wavelengths or spectra used to generate the acoustic data. The data processing circuitry (such as processor 30) may derive one or more physiological characteristics based on data generated by the photoacoustic sensor 12. For example, based at least in part upon data received from the acoustic detector 20, the processor 30 may calculate the amount or concentration of a constituent of interest in a localized region of tissue or blood using various algorithms. In one embodiment, these algorithms may use coefficients, which may be empirically determined, that relate the detected acoustic waves generated in response to pulses of light at a particular wavelength or wavelengths to a given concentration or quantity of a constituent of interest within a localized region.

In one embodiment, processor 30 may access and execute coded instructions from one or more storage components of the monitor 12, such as the RAM 34, the ROM 60, and/or the mass storage 62. For example, code encoding executable algorithms may be stored in a ROM 60 or mass storage device 62 (such as a magnetic or solid state hard drive or memory or an optical disk or memory) and accessed and operated according to processor 30 instructions. Such algorithms, when executed and provided with data from the sensor 10, may calculate a physiological characteristic as discussed herein (such as the concentration or amount of a constituent of interest). Once calculated, the physiological characteristic may be displayed on the display 14 for a caregiver to monitor or review.

With the foregoing system discussion in mind, light emitted by the light source 18 of the photoacoustic sensor 10 may be used to generate acoustic signals in proportion to the amount of an absorber (e.g., a constituent of interest, such as hemoglobin) in a targeted localized region. To that end, certain embodiments of the disclosure include photoacoustic sensors 10 with features that direct light into a patient's tissue and reduce absorption by the acoustic detector 20 and other sensor structures.

As discussed, the diffuser 22 may diffuse light from the light source 18 if the sensor 10 is detached from the patient while the light source 18 is in operation. When the sensor 10 is applied to the patient, the light is emitted into the tissue from the light source 18 and any light directing elements associated with the sensor to direct the light source to a desired focal spot. The diffuser 22 as provided may be integrated into the sensor 12 as a unitary assembly, such that the diffuser 22 is not removable from the sensor 10.

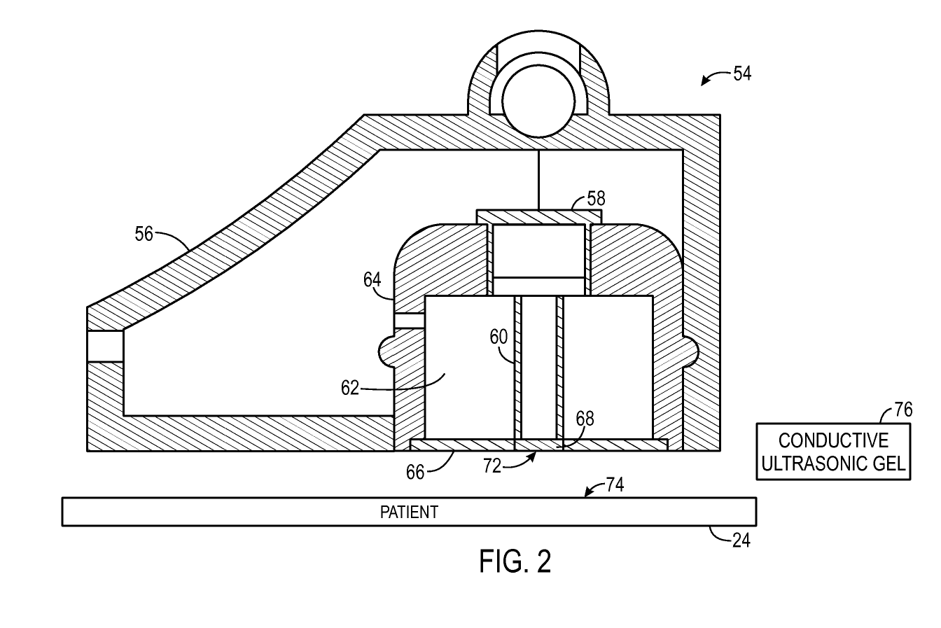

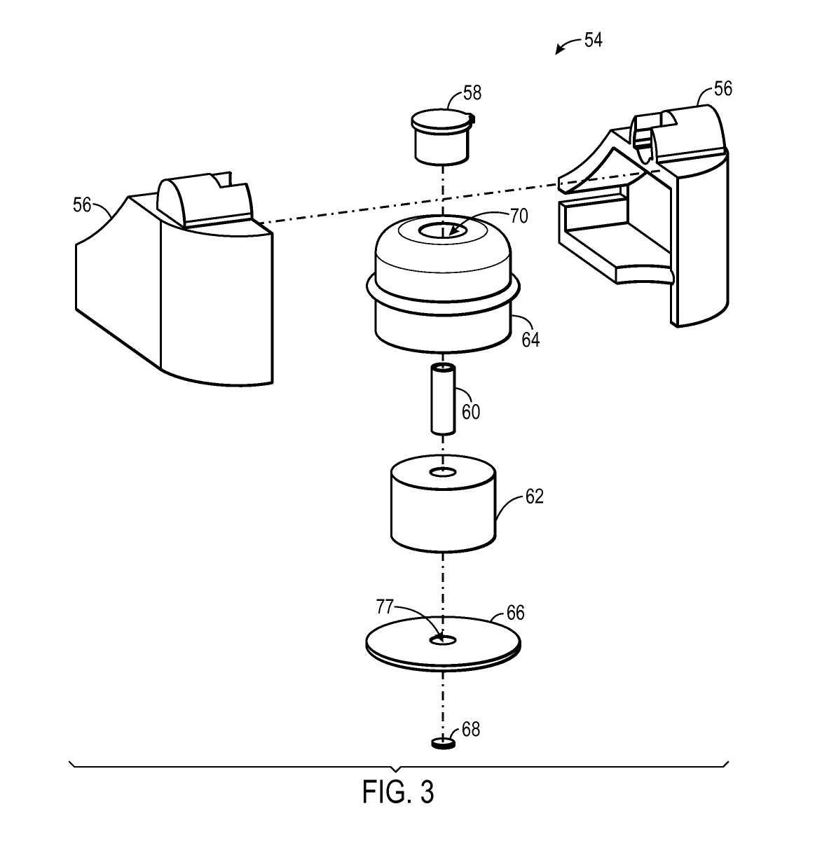

To that end, certain disclosed embodiments of the present techniques provide sensor assemblies that position and/or arrange a light diffusing element (e.g., diffuser 22) with respect to the light source 18 to yield the desired result of light diffusion when the sensor 10 is not applied to the patient when the light source 18 is in operation. For example, FIGS. 2 and 3 illustrate an embodiment of a photoacoustic sensor assembly 54 including a housing or holder 56, a laser diode 58, an optical fiber 60, an ultrasound transducer 62, a fixture 64, a Rexolite cover 66, and a diffuser 68. In certain embodiments, a reflective coating may be disposed on the ultrasound transducer and/or on the optical fiber to increase the signal to noise ratio of the received signals, as described in more detail below.

In the illustrated embodiment, the fixture 64 functions as a housing that encloses the ultrasound transducer 62 and the optical fiber 60. The ultrasound transducer 62 includes an aperture 70 sized and shaped to receive the optical fiber 60 such that the optical fiber 60 is retained in the ultrasound transducer 62, for example, via an interference fit. The laser diode 58 is coupled to the optical fiber 60 such that the light emitted by the laser diode 58 is transmitted into the optical fiber 60. In an alternative arrangement, the laser diode 58, recessed relative to an exterior surface of the assembly 54, may direct light directly through the aperture 70, which may be coated to function as a light pipe. Further, to achieve a desired focal intensity with a sensor assembly that does not unduly protrude from the patient when applied, the optical fiber may be selected to be relatively short in length, e.g., 20 mm or less. The optical fiber 60 may also be selected to achieve a desired beam size, which may be at least partially dependent on the fiber size (i.e., fiber diameter) and any further light guiding components or openings at the patient-contacting surface.

In addition, a reflective coating or cover may be applied to the ultrasound transducer 62 to reduce any unwanted optical crosstalk due to reflected light striking the transducer surface. In certain embodiments, this crosstalk may obscure the intended ultrasound signal from the probed tissue if the reflective coating or covering is not provided. The reflective coating may be, but is not limited to, vacuum deposited reflective material, adhesive backed film, or any other suitable reflective coating, as determined by implementation-specific considerations. In certain embodiments, the reflective coating will also mitigate any light scattered from the fiber optic diffuser 68 that may create crosstalk on the ultrasound transducer 62. Also, a reflective coating may be applied to a portion of the optical fiber 60 that is located inside the ultrasound transducer 62 or protrudes from the front of the transducer 62. Again, in some embodiments, this reflective coating may mitigate optical crosstalk from light scattered from the diffuser on the fiber optic cable.

In the illustrated embodiment, the Rexolite cover 66 is adhered to the ultrasound transducer 62, but in other embodiments, the Rexolite cover 66 may be assembled with the other components of the sensor 54 in any desired manner. Further, in the described embodiment, the Rexolite cover 66 is utilized as a spacer because of its low ultrasound attenuation and its ability to be machined to the desired shape, which facilitates tuning of the direction of ultrasound propagation during operation of the PA Sensor 54. However, in other embodiments, any desired spacer having any desired features may be utilized, not limited to Rexolite, depending on implementation-specific considerations. For instance, in some embodiments, the Rexolite cover 66 may be replaced with any material having a low ultrasound impedance (i.e., an ultrasound impedance approximately equal or close to the ultrasound impedance of the tissue of the patient) including an adhesive used to attach the sensor to a patient's skin. For example, in one embodiment, the ultrasound impedance of the spacer may be approximately 1.5-1.6 MRayls. The thickness of the spacer 66 may also influence the size of the emitted beam, depending on the distance from the fiber outlet to the subject's skin. For example, a thicker spacer 66 may result in a larger spot.

During operation of the sensor 54, the laser diode 58 transmits light into the optical fiber 60. The optical fiber 60 guides the light emitted by the laser diode 58 and prevents the beam from dispersing, thus providing a higher light density beam when the beam reaches the surface of the patient's tissue. The diffuser 68 diffuses the guided light at a diffusing angle, and the light is emitted toward the patient 24. When in use, a surface or interface 72 of the sensor 54 comes into contact with a surface or interface 74 of the patient 24. Therefore, the emitted light is transmitted into the patient 24 to probe features of the patient's anatomy. The ultrasound transducer 70, which is the acoustic detector in the illustrated embodiment, detects PA signals that are generated by a heating and thermal expansion effect within the interrogation region of the patient 24. These signals are then utilized for one or more downstream medical applications.

In one embodiment, the diffuser 68 is integrated into the Rexolite spacer 66 and adhered to the tip of the optical fiber 60 from which the light exits before reaching the patient. For example, as shown in FIG. 3, the spacer 66 may include an opening 77 through the spacer into which the diffuser 68 is integrated, either via adhesion or other means. Alternatively, the diffuser may be integrally formed with the spacer 66 (e.g, the spacer 66 and the diffuser 68 may be formed as a single piece, with the diffuser portion being only a part of the spacer 66. Such an embodiment may be formed by treating only a portion of the spacer 66 or coating only a portion of the spacer with a material that forms the diffuser 68. In the depicted embodiment, the spacer 66 is in the form of a disk that slots into a complementary space formed in the housing 56. In this manner, the diffuser 68 may be aligned with the optical fiber 60 when incorporated with the spacer 66 into the sensor 54. Further, recessing the spacer 66 and/or the diffuser 68 within the housing 56 positions the lateral end (e.g., an outer circumference in embodiments in which the spacer 66 and/or diffuser 68 is a disk) of the spacer 66 within the housing such that light-absorbing portions of the housing 56 absorb any light diffused laterally during operation.

In alternative embodiments, the diffuser 68 may be incorporated into the sensor 54 in a variety of other ways. For example, the diffuser 68 may be an oversized cylinder adapted to be placed over the tip of the optical fiber 60. Alternatively, the diffuser 68 may be deposited as diffusing material onto the tip of the optical fiber 60, or integrated into the tip of the optical fiber 60 during manufacturing. Still further, the diffuser 68 may be arranged within the housing 56 such that the diffuser 68 abuts the tip of the optical fiber 60 when the sensor 54 is assembled (e.g., via adherence to the Rexolite cover 66 and proper positioning within the housing 56). In other embodiments, the diffuser 68 may replace the Rexolite cover 66. Additionally, it should be noted that the diffuser 68 may be positioned internal to the housing 56 such that the diffuser 68 does not come into direct contact with the patient's tissue during use, or external to the housing 56 such that the diffuser 68 is exposed to the surrounding environment and may come into contact with the patient's tissue during use. That is, the diffuser 68 may be positioned on one or more surfaces of the spacer 66. Indeed, the positioning of the diffuser 68 within the sensor 54 is not limited to the placements shown and described herein and may be placed in any suitable location, depending on implementation-specific considerations. In one embodiment, the diffuser 68 may be formed as a coating or layer on all or part of the spacer 66. For example, if the diffuser 68 forms an area larger than an exit port of the optical fiber 60, the alignment tolerance of the diffuser 68 and the optical fiber 60 may be improved. Accordingly, in certain embodiments, the diffuser 68 abuts the optical fiber and has a surface area at least 2×, 5×, or 10× the surface area formed by the cross-section (i.e. exit port) of the optical fiber 60. In another embodiment, the diffuser 68 may cover or be coated on at least one surface of the spacer 66. Because the diffuser 68 does not affect acoustic wave detection, the diffuser 68 may also contact or be adjacent to the transducer 62.

In certain embodiments, a conductive ultrasonic gel 76 is placed on the surface 74 of the patient's tissue, the surface 72 of the sensor 54, or both to facilitate the transmission of the ultrasonic waves between the sensor 54 and the patient 24. That is, it may be desirable to utilize the conductive ultrasonic gel 76 in some embodiments to provide a conductive medium between the sensor 54 and the patient 24 that functions as an ultrasound coupling material. In embodiments in which the gel 76 is utilized, the intensity of the light that is emitted by the sensor 54 can be intensified if the gel forms a condensing lens shape. Further, by providing the diffuser 68, the beam spot produced may be of increased uniformity, which reduces the peak intensity. Providing the diffuser 68 as a wide angle diffuser (i.e., having a diffusing angle greater than or equal to 80 degrees) enables proper functioning of the sensor 54 with a reduced laser classification (as compared to the laser classification that would result from use of ultrasonic gel without the wide angle diffuser) despite the use of the ultrasonic gel 76.

For example, in some embodiments, the angle of the diffuser 68 may be selected so that the light emitted by the laser diode 58 is diffused at an angle wide enough such that the light emitted via sensor interface 72 enables the sensor 54 to be classified as Class 1 according to the IEC 60825-1 standard when the interface 72 is opposed by air and as a Class 1 when the interface 72 is ultrasonic gel 76 and the ultrasonic gel 76 is opposed by air. In certain embodiments, the laser diode 58 may emit light that enables the laser diode 58, taken alone outside of the sensor 54, to be classified as Class 3B according to the IEC 60825-1 standard. In such embodiments, by embedding the laser diode 58 in the sensor 54 and providing a wide angle diffuser 68 at the tip of the optical fiber 60, the classification of the sensor 54 may be reduced, thus making the sensor 54 usable in a clinical setting, while the optical power and power density of the laser diode 58 is maintained.

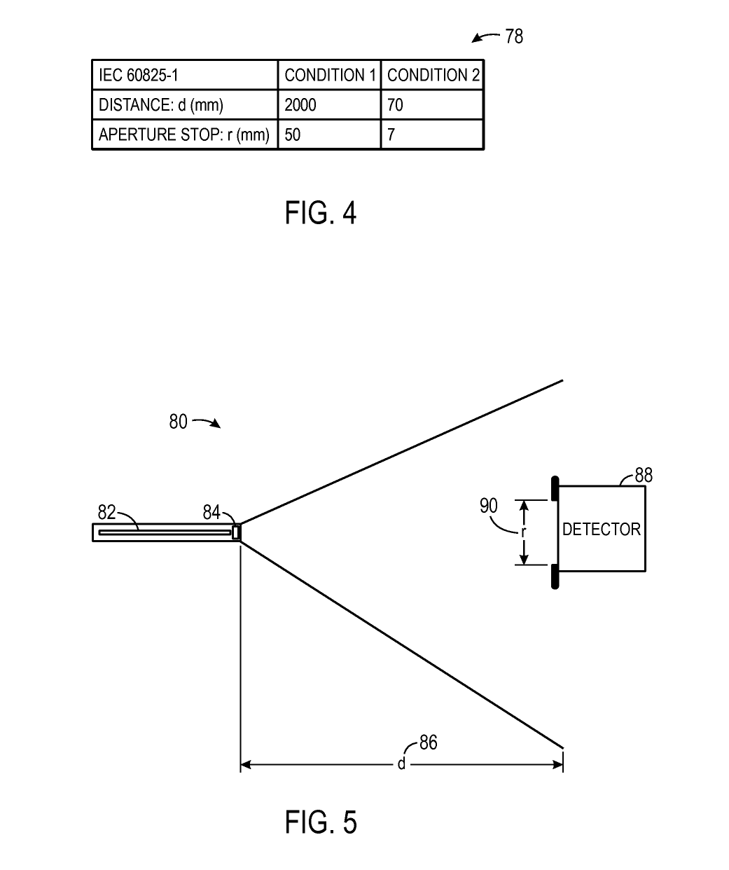

FIG. 4 is a chart 78 showing the measurement setups specified by the IEC 60825-1 standard. When classifying a device according to the IEC 60825-1 standard, each of the conditions set forth in chart 78 is tested in accordance with the setup 80 illustrated in FIG. 5, and the measured energy is recorded. For example, as shown in FIG. 5, the setup 80 includes a light emitting device 82 positioned to emit light from a light emitting point 84 at a distance 86 from a detector 88 having an aperture stop 90 (with highest intensity detected at detector 88). To test condition one set out in table 78, the light emitting point 84 is positioned such that the distance 86 is 2000 mm, and the aperture stop 90 is set at 50 mm. Likewise, to test condition two, the distance 86 is set to 70 mm, and the aperture stop 90 is set to 7 mm.

Once the energy is measured at each of the conditions set forth in table 78, the energy measured at each condition is used to classify the emitting device 82. Specifically, each measurement at each of the conditions in table 78 is compared to an AEL for that condition. That is, by comparing the measured energy to calculated thresholds associated with each class in the IEC 60825-1 standard and the device being analyzed, the device 82 may be classified. The calculated thresholds for a given embodiment are determined based on features of the optical system being analyzed (e.g., based on pulsed frequency of light, beam spot size, use of diffuser, wavelength, etc.). For example, for the embodiment illustrated in FIGS. 2 and 3 that includes a repetitively pulsed laser, the AEL to which the measured energy is compared is determined by calculating a plurality of AELs and selecting the most restrictive AEL for comparison. It should be noted that the particular AEL to which the measured energy is compared depends on implementation-specific design considerations, such as the type of laser used in the device.

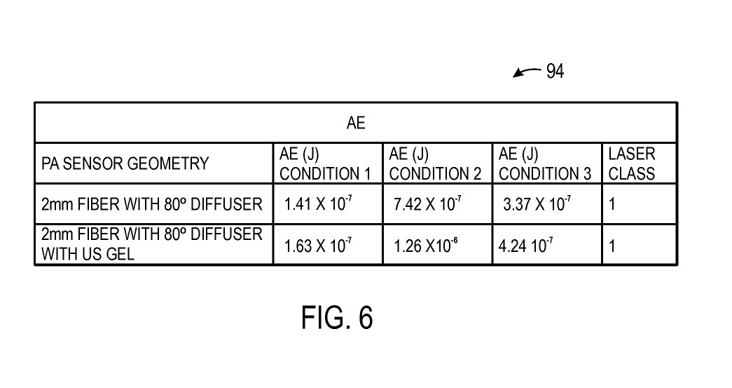

FIG. 6 is a table 94 illustrating experimental results obtained when an embodiment of the sensor 54 was tested according to the IEC 60825-1 standard, as set forth in FIGS. 4-5. In this embodiment, a 2 mm optical fiber was provided with a wide angle diffuser having a diffusing angle of 80 degrees positioned on the optical fiber tip. The diffuser was a thin (0.005″) film polycarbonate holographic relief cut to a 2 mm diameter and glued to the optical fiber tip using index refraction (for low reflection) and low viscosity (for thin layer) adhesive. The optical fiber was a plastic fiber formed from poly(methyl methacrylate) (PMMA). The laser (905 nm) was pulsed at 500 Hz with a peak power of 250 W and pulse width of 100 ns. The table 94 illustrates experimental results obtained both with and without the use of ultrasonic gel. In some embodiments in which ultrasonic gel is used, the gel may form a lens that condenses the beam (i.e., reduces the angle of the beam, thereby producing a higher intensity). However, for the embodiment illustrated in FIGS. 2 and 3, the apparent spot size is larger than the 2 mm fiber.

Without the diffuser, the laser beam exits the fiber tip to air at a half angle of approximately 23 degrees, which is based on the numerical aperture (N.A.=0.4) of the fiber. When the measurement for condition 2 was taken without the diffuser, the highest measured energy level resulted in classification of the sensor 54 in Class 3R. However, when the diffuser was affixed to the tip of the optical fiber and the energy was measured in accordance with condition 2 of table 78, the sensor 54 was classified in Class 1. Further, when the sensor 54 was tested in combination with conductive ultrasonic gel 76, the sensor 54 was classified in Class 1. Accordingly, by incorporating a wide angle diffuser into sensor 54, the classification of the sensor 54 may be reduced as compared to the classification of the laser diode 58 not incorporated into the assembly, thereby rendering the sensor 54 suitable for use in a clinical setting.

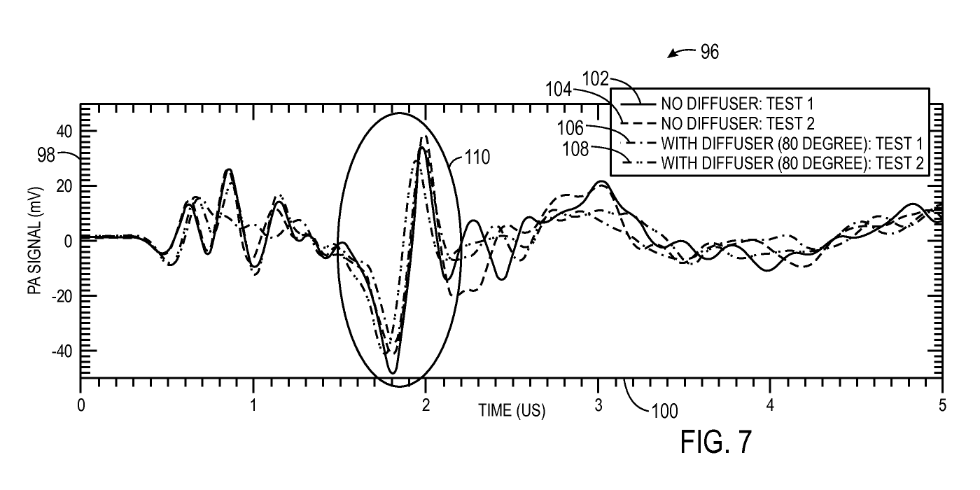

Still further, the sensor 54 was experimentally tested to determine any possible effects the presence of the wide angle diffuser would have on acquired photoacoustic measurements. To that end, the sensor 54 was activated when placed over a patient's superficial temporal artery (STA), and the patient's PA signal was recorded. The sensor 54 was tested both with and without the 80 degree diffuser, and the results are shown in FIG. 7. Specifically, FIG. 7 shows a plot 96 have a PA signal axis 98 and a time axis 100. The patient was probed with the sensor not including the diffuser twice, and the resulting signals 102 and 104 are shown. The patient was also probed twice with the sensor including the 80 degree diffuser, and the resulting signals 106 and 108 are shown. As illustrated, the patient's STA signal, as indicated by the region 110 of the plot 96, was approximately the same with or without the diffuser incorporated into the sensor 54. In certain embodiments, a sensor 10 may be configured to include configurations such as those used to achieve the STA signal with appropriate diffusing of the laser light when the sensor is detached from the patient. That is, when the sensor 10 is operated to activate the light source 18 and applied to the patient, an STA signal may be generated and provided to the monitor 12. When the sensor is detached from the patient and the light source 18 is in operation, the light is diffused such that the light source may be classified as a Class 1 laser. Accordingly, by incorporating the wide angle diffuser into the sensor assembly, the classification of the assembly may be reduced while the ability of the sensor to obtain clinically desirable measurements may be preserved.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Further, it should be understood that certain elements of the disclosed embodiments may be exchanged and/or combined with one another. Rather, the various embodiments may cover all modifications, equivalents, and alternatives falling within the spirit and scope of the disclosure as defined by the following appended claims.

What is claimed is:

1. A photoacoustic system, comprising:
a sensor, comprising:
a body;
one or more light emitting components disposed in the body and configured to emit one or more wavelengths of light;
a light guide configured to receive the one or more wavelengths of light and guide the one or more wavelengths of light into an interrogation region of a patient;
a wide angle diffusing element disposed on a tip of the light guide and configured to diffuse the one or more wavelengths of light when the sensor is opposed by air; and
one or more acoustic detectors disposed in the body and configured to detect acoustic energy generated by the interrogation region of the patient in response to the emitted light when the sensor is opposed by the patient.

2. The photoacoustic system of claim 1, comprising a patient monitor communicatively coupled to the sensor and configured to receive a signal from the one or more acoustic detectors that corresponds to the detected acoustic energy.

3. The photoacoustic system of claim 1, comprising an optically transparent spacer disposed between the one or more light emitting components and the patient when the sensor is applied to the patient.

4. The photoacoustic system of claim 1, wherein the one or more light emitting components comprises a laser diode, the light guide comprises an optical fiber, and the one or more acoustic detectors comprises an ultrasound transducer.

5. The photoacoustic system of claim 4, wherein the laser diode is coupled to an end portion of the optical fiber, and the optical fiber is disposed in a central aperture formed through the ultrasound transducer.

6. The photoacoustic system of claim 1, wherein the wide angle diffusing element comprises a diffusing angle of greater than or equal to 80 degrees.

7. A photoacoustic sensor, comprising:
a light emitting component configured to emit one or more wavelengths of light into an interrogation region of a patient;
an acoustic detector configured to detect acoustic energy generated by the interrogation region of the patient in response to the emitted light;
an interface through which the emitted light is configured to pass into the interrogation region of the patient; and
a diffusing element configured to diffuse the emitted light at an angle wide enough that the emitted light enables the photoacoustic sensor to be classified as Class 1 according to the IEC 60825-1 standard when the interface is opposed by air and to be classified as Class 1 according to the IEC 60825-1 standard when the interface has conductive ultrasonic gel and is opposed by air.

8. The photoacoustic sensor of claim 7, wherein the light emitting component comprises a laser diode, and the emitted light enables the laser diode to be classified as Class 3B according to the IEC 60825-1 standard.

9. The photoacoustic sensor of claim 7, wherein the diffusing element diffuses the emitted light at an angle greater than 80 degrees.

10. The photoacoustic sensor of claim 7, comprising a spacer disposed between the light emitting component and the patient when the interface is opposed by the patient.

11. The photoacoustic sensor of claim 10, wherein the diffusing element is disposed on the spacer.

12. The photoacoustic sensor of claim 10, wherein the diffusing element is integrally formed with the spacer such that the light diffusing element is only a portion of the spacer.

13. The photoacoustic sensor of claim 7, comprising a light guide coupled at a first end of the light guide to the light emitting component and configured to receive the emitted light and to guide the emitted light into the interrogation region of the patient when the interface is opposed by the patient.

14. The photoacoustic sensor of claim 13, wherein the light guide is located in a channel axially disposed through the acoustic detector, and the diffusing element is disposed at a second end of the light guide.

15. The photoacoustic sensor of claim 7, wherein the acoustic detector comprises an ultrasound transducer.

16. A photoacoustic sensor, comprising:
a housing configured to be applied to a patient and comprising a patient-contacting surface;
a laser light emitting component recessed within in the housing and configured to emit one or more wavelengths of light;
a light guide configured to receive the one or more wavelengths of light and direct the light into the patient via emission through the patient-contacting surface;
a wide angle diffusing element disposed on a tip of the light guide and configured to diffuse the one or more wavelengths of light when the sensor is opposed by air;
one or more acoustic detectors disposed in the housing and configured to detect acoustic energy generated by the interrogation region of the patient in response to the emitted light when the sensor is applied to the patient; and
a spacer separating the acoustic detector from the patient-contacting surface.

17. The photoacoustic sensor of claim 16, wherein the spacer separates the diffusing element from the patient-contacting surface.

18. The photoacoustic sensor of claim 16, wherein the housing comprises an opening configured to receive the spacer such that the spacer is flush with the patient-contacting surface of the housing.

19. The photoacoustic sensor of claim 16, wherein lateral ends of the spacer abut portions of the housing that are configured to absorb the one or more wavelengths of light.

20. The photoacoustic sensor of claim 16, wherein the diffuser is disposed on a first surface of the spacer that opposes a second surface that is configured to contact the patient.

21. The photoacoustic sensor of claim 20, wherein the diffuser covers a majority of the first surface of the spacer.

22. The photoacoustic sensor of claim 16, wherein the wide angle diffusing element comprises a diffusing angle of greater than or equal to 80 degrees.
